(19) United States
(12) Patent Application Publication
(10) Pub. No.: US 2012/0136257 A1
(43) Pub. Date: May 31, 2012

(54) SNR Through Ambient Light Cancellation

(75) Inventors: Kalpathy Krishnan, Boulder, CO (US); Tom Wilmering, Westminster, CO (US); Thomas Geske, Erie, CO (US)

(73) Assignee: Nellcor Puritan Bennett LLC, Boulder, CO (US)

(21) Appl. No.: 12/956,062

(22) Filed: Nov. 30, 2010

(51) Int. Cl. A61B 6/00

(52) U.S. Cl. 600/476

(57) ABSTRACT

Systems, methods, and devices for improved patient monitor signal processing with higher signal-to-noise ratio (SNR) are provided. In accordance with an embodiment, an electronic patient monitor may include drive circuitry, a current-to-voltage converter, and feedback circuitry. The drive circuitry may drive an emitter of a medical sensor with dark periods during which the emitter does not emit light, and the current-to-voltage converter may receive and amplify a photocurrent signal from a detector of the sensor. The feedback circuitry may provide a feedback signal to the current-to-voltage converter. The feedback signal, based at least in part on the output of the current-to-voltage converter during the dark periods, may cause the current-to-voltage converter to substantially exclude an ambient light component of the photocurrent. As a result, the current-to-voltage converter may employ a higher transimpedance without distorting the output voltage signal due to oversaturation, and thus may achieve a higher SNR.

### BACKGROUND

The present disclosure relates generally to medical monitoring systems and, more particularly, to non-invasive medical monitoring systems employing optical sensors.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide variety of devices have been developed for non-invasively monitoring physiological characteristics of patients. For example, a pulse oximetry sensor system may detect various patient blood flow characteristics, such as the blood-oxygen saturation of hemoglobin in arterial blood, the volume of individual blood pulsations supplying the tissue, and/or the rate of blood pulsations corresponding to each heart beat of a patient. To determine these physiological characteristics, light may be emitted into patient tissue, where the light may be scattered and/or absorbed in a manner dependent on such physiological characteristics.

Non-invasive medical sensor systems may include a medical sensor and an electronic patient monitor. The monitor may send driving signals to an emitter in the sensor, causing the sensor to emit light into pulsatile patient tissue. A detector in the medical sensor may detect the light after it has passed through the patient tissue, generating an electrical current proportional to the amount of detected light. This electrical current, referred to as a photocurrent, may be received by the patient monitor and converted into a voltage signal using a current-to-voltage (I-V) converter. The resulting voltage signal subsequently may be analyzed to determine certain physiological characteristics of the patient tissue.

When the I-V converter transforms the photocurrent from the photodetector to a voltage signal, thermal noise, also known as Johnson noise, may arise. The Johnson noise may be proportional to the square root of a transimpedance employed by the I-V converter, while the signal gain of the I-V converter may be directly proportional to the transimpedance. As a result, the higher the transimpedance, the lower the signal-to-noise ratio (SNR) of the I-V converter based on Johnson noise (e.g., when the transimpedance increases by a factor of ten, the SNR improves by a factor of \sqrt{10}). On the other hand, the higher gain brought about by the higher transimpedance may cause the I-V converter to amplify the photocurrent beyond a signal saturation region of the I-V converter, which may produce a distorted output voltage signal.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

Embodiments of the present disclosure relate to systems, methods, and devices for improved patient monitor signal processing with higher signal-to-noise ratio (SNR). In accordance with an embodiment, an electronic patient monitor may include drive circuitry, a current-to-voltage converter, and feedback circuitry. The drive circuitry may drive an emitter of a medical sensor with dark periods during which the emitter does not emit light, and the current-to-voltage converter may receive and amplify a photocurrent signal from a detector of the sensor. The feedback circuitry may provide a feedback signal to the current-to-voltage converter. The feedback signal, based at least in part on the output of the current-to-voltage converter during the dark periods, may cause the current-to-voltage converter to substantially exclude an ambient light component of the photocurrent. As a result, the current-to-voltage converter may employ a higher transimpedance without distorting the output voltage signal due to oversaturation, and thus may achieve a higher SNR.

### BRIEF DESCRIPTION OF THE DRAWINGS

Various aspects of this disclosure may be better understood upon reading the following detailed description and upon reference to the drawings in which:

FIG. 1 is a perspective view of a non-invasive medical sensor system, in accordance with an embodiment;

FIG. 2 is a block diagram of the medical sensor system of FIG. 1, in accordance with an embodiment;

FIG. 3 is a timing diagram representing emitter excitation that may be employed by the sensor system of FIG. 1, in accordance with an embodiment;

FIG. 4 is a schematic circuit diagram representing a current-to-voltage (I-V) converter employed in the system of FIG. 1, in accordance with an embodiment;

FIG. 5 is a flowchart describing an embodiment of a method for eliminating ambient light noise through the I-V converter of FIG. 4;

FIG. 6 is a flowchart describing another embodiment of a method for eliminating ambient light noise through the I-V converter of FIG. 4;

FIG. 7 is a plot modeling a photocurrent obtained from a detector of the medical sensor system of FIG. 1, in accordance with an embodiment;

FIG. 8 is a plot modeling an output voltage signal of the I-V converter of FIG. 4 when the ambient light cancellation techniques described herein are not employed; and

FIG. 9 is a plot modeling an output voltage signal of the I-V converter of FIG. 4 when the ambient light cancellation techniques described herein are employed, in accordance with an embodiment.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Present embodiments relate to medical sensor systems for non-invasively monitoring physiological patient characteristics. These systems may involve emitting light through patient tissue using an emitter and detecting an amount of light scattered by the patient tissue using a photodetector. The photodetector may generate a photocurrent, which may converted to an output voltage signal for use by an electronic patient monitor using a current-to-voltage (I-V) converter in the monitor. Such an output voltage signal may be analyzed to obtain physical parameters of the patient, including the blood-oxygen saturation of hemoglobin in arterial blood, the volume of individual blood pulsations supplying the tissue, and/or the rate of blood pulsations corresponding to each heart beat of a patient.

In certain embodiments, the I-V converter of the patient monitor may be a transimpedance amplifier with a signal-to-noise ratio (SNR) that improves with respect to thermal noise (e.g., Jolmson noise) as a transimpedance of the I-V converter increases. Indeed, although such thermal noise may be proportional to the square root of the transimpedance, the signal gain is directly proportional to the transimpedance. Thus, increasing the transimpedance by a factor of 10 (e.g., from 100 kΩ to 1 MΩ) may improve the SNR of the I-V converter by a factor of \sqrt{10}, or approximately 3.

Such a higher gain may amplify the photocurrent beyond a saturation region of the I-V converter if the entire photocurrent is amplified and the output voltage signal of the I-V converter may be distorted. Specifically, certain components of interest of the photocurrent may correspond to emitted light that passes through or is reflected by the patient tissue, but other components of the photocurrent may correspond to ambient light noise (e.g., lighting in a patient room). To ensure that the components of interest rather than the components of ambient light noise of the photocurrent are amplified by the I-V converter, present embodiments employ ambient light cancellation circuitry to provide a baseline for the I-V converter through a feedback mechanism.

In particular, during periods when the emitter of the medical sensor is not excited and therefore not emitting light into the patient, referred to as “dark periods”, the photocurrent signal may include substantially only the components corresponding to ambient light. During these dark periods, the output voltage signal of the I-V converter may be monitored to determine the amount of ambient light being detected by the detector of the medical sensor. Based on the amount of ambient light, a feedback signal may be applied to the I-V converter, which may cause the I-V converter to amplify substantially only the components of interest of the photocurrent during the periods of time when the emitter of the medical sensor is excited. In this way, the I-V converter may avoid amplifying the components of interest of the photocurrent beyond a saturation region of the I-V converter when the I-V converter employs a higher transimpedance, and thus realizes a higher signal gain. Since the I-V converter may operate with a significantly higher SNR, the components of interest of the photocurrent may be smaller in magnitude. Accordingly; medical sensors used in a such medical sensor system may be designed using less powerful emitters and/or detectors, reducing costs, and/or medical sensor emitters may emit less light, improving patient comfort.

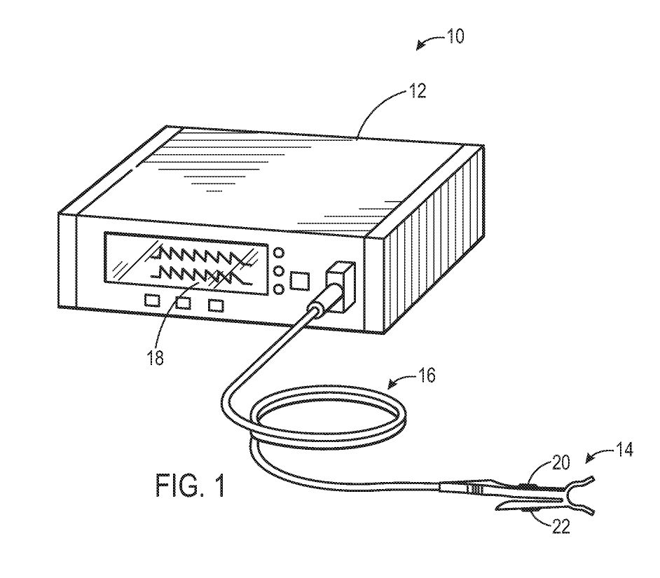

With the foregoing in mind, FIG. 1 illustrates a perspective view of an embodiment of a non-invasive medical sensor system 10 involving an electronic patient monitor 12 and a medical sensor 14. Although the embodiment of the system 10 illustrated in FIG. 1 relates to pulse oximetry, the system 10 may be configured to obtain a variety of physiological measurements. For example, the system 10 may, additionally or alternatively, measure water fraction of tissue or perform other non-invasive medical monitoring techniques.

The patient monitor 12 may exchange signals with the medical sensor 14 via a communication cable 16. The patient monitor 12 may include a display 18, a memory, and various monitoring and control features. In certain embodiments, the patient monitor 12 may include a processor that may determine a physiological parameter of a patient based on these signals obtained from the medical sensor 14. Indeed, in the presently illustrated embodiment of the system 10, the medical sensor 14 is a pulse oximetry sensor that may non-invasively obtain pulse oximetry data from a patient. In other embodiments, the medical sensor 14 may represent any other suitable non-invasive optical sensor.

The medical sensor 14 may attach to pulsatile patient tissue (e.g., a patient's finger, ear, forehead, or toe). In the illustrated embodiment, the medical sensor 14 is configured to attach to a finger. An emitter 20 and a detector 22 may operate to generate non-invasive pulse oximetry data for use by the patient monitor 12. In particular, the emitter 20 may transmit light at certain wavelengths into the tissue and the detector 22 may receive the light after it has passed through or is reflected by the tissue. The amount of light and/or certain characteristics of light waves passing through or reflected by the tissue may vary in accordance with changing amounts of blood contingents in the tissue, as well as related light absorption and/or scattering.

The emitter 20 may emit light from two or more light emitting diodes (LEDs) or other suitable light sources into the pulsatile tissue. The light that is reflected or transmitted through the tissue may be detected using the detector 22, which may be a photodetector (e.g., a photodiode), once the light has passed through or has been reflected by the pulsatile tissue. When the detector 22 detects this light, the detector 22 may generate a photocurrent proportional to the amount of detected light, which may be transmitted through the cable 16 to the patient monitor 12. As described in greater detail below, the patient monitor 12 may convert the photocurrent from the detector 22 into a voltage signal that may be analyzed to determine certain physiological characteristics of the patient.

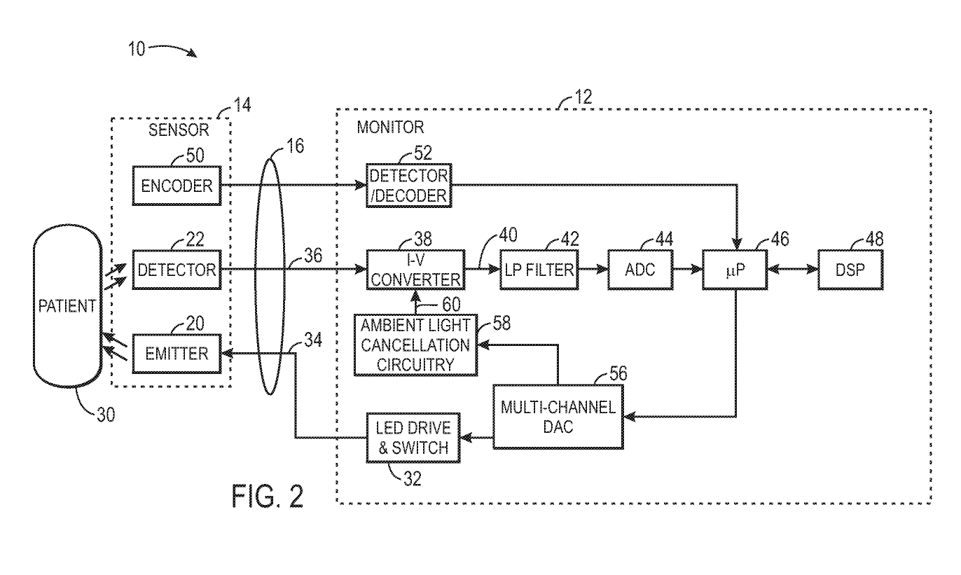

As illustrated in FIG. 2, which describes the operation of the medical sensor system 10 in greater detail, the emitter 20 may emit light into a patient 30, which may be reflected by or transmitted through patient 30 and detected by the detector 22. An LED drive and/or switch 32 may generate LED driving signals (e.g., LED current signals 34) to cause the LEDs of the emitter 20 to become excited and emit the light into the patient 30. In certain embodiments, the LED current signals 34 may include red wavelengths of between approximately 600-700 nm and/or infrared wavelengths of between approximately 800-1000 nm. In some embodiments, the LEDs of the emitter 20 may emit three or more different wavelengths of light. Such wavelengths may include a red wavelength of between approximately 620-700 nm (e.g., 660 nm), a far red wavelength of between approximately 690-770 nm (e.g., 730 nm), and an infrared wavelength of between approximately 860-940 nm (e.g., 900 nm). Other wavelengths may include, for example, wavelengths of between approximately 500-600 nm and/or 1000-1100 nm. Regardless of the number and wavelength of LEDs driven by the LED drive and/or switch 32, the LED current signals 34 may include at least one “dark period” during which no LEDs of the emitter 20 are being driven. During such dark periods, the emitter 20 may not emit any light into the patient 30 tissue.

The detector 22 may detect the emitted light that passes through or is reflected by the tissue of the patient 30 during the non-dark periods, generating a photocurrent 36 that varies depending on the amount and wavelength of light emitted by the emitter 20 and the various physiological characteristics of the patient 30. In addition, the detector 22 may also generate a component of the photocurrent 36 in response to ambient light near the patient 30 (e.g., room lighting or light from windows). In general, such ambient light may be present at all times while the sensor 14 is attached to the patient 30. In certain situations, such as when the patient 30 is in relatively intense light (e.g., if the patient is outdoors or under a surgical light), a greater part of the photocurrent 36 may be due to the ambient light than to the light passing through or reflected by the patient 30.

A current-to-voltage (I-V) converter 38 may convert the photocurrent 36 from the detector 22 into an output voltage signal 40, as discussed further below. The output voltage signal 40 may be filtered in a low pass (LP) filter 42 before being digitized in an analog-to-digital converter (ADC) 44 and received by a microprocessor 46. The microprocessor 46, which may be a microcontroller (e.g., a PIC microcontroller), may perform certain processing operations based on the received data. In some embodiments, the microprocessor 46 may transfer certain data to another microprocessor, such as a digital signal processor (DSP) 48, which may determine certain physiological parameters of the patient 30.

In certain embodiments, the medical sensor 14 may also include an encoder 50 that may provide signals indicative of the wavelength of one or more light sources of the emitter 20, which may allow for selection of appropriate calibration coefficients for calculating a physical parameter such as blood oxygen saturation. Some embodiments of the encoder 50 may indicate a propensity of the medical sensor 14 to detect ambient light to assist the patient monitor 12 in determining the feedback signal to provide to the I-V converter 38. For example, the encoder 50 may provide an offset voltage representing a typical ambient light voltage, which may serve as an initial starting voltage of the feedback signal. The encoder 50 may, for instance, be a coded resistor, EEPROM or other coding devices (such as a capacitor, inductor, PROM, RFID, parallel resident currents, or a colorimetric indicator) that may provide a signal to the microprocessor 46 related to the characteristics of the medical sensor 14 to enable the microprocessor 46 to determine the appropriate calibration characteristics of the medical sensor 14. Further, the encoder 50 may include encryption coding that prevents a disposable part of the medical sensor 14 from being recognized by a microprocessor 46 unable to decode the encryption. For example, a detector decoder 52 may be required to translate information from the encoder 50 before it can be properly handled by the processor 46. In seine embodiments, the encoder 50 and/or the detector decoder 52 may not be present.

As mentioned above, a portion of the photocurrent 36 from the detector 22 may be due to ambient light detected by the detector 22. If the I-V converter 38 simply converted the entire photocurrent signal 36 into an output voltage signal 40 without accounting for components of the photocurrent signal 36 that correspond to the ambient light, the output voltage signal 40 could saturate and become distorted or the gain of the converter 38 could be reduced to prevent such saturation, improving the SNR. To prevent signal distortion resulting from output voltage signal 40 saturation and/or to improve the converter 38 SNR, the patient monitor 12 may include certain circuitry to cancel the effect of ambient light on the photocurrent 36.

In particular, the microprocessor 46 may sample the output voltage signal 40, after being filtered in the LP filter 42 and digitized by the ADC 44, during dark periods when the emitter 20 is not emitting any light. During such dark periods, substantially all of the photocurrent signal 36 may arise due to ambient light. Accordingly, the output voltage signal 40 obtained during the dark periods may also substantially only correspond to ambient light detected by the detector 22. The resulting output voltage signal 46 obtained during dark periods may be used to cancel out the ambient light component of the photocurrent signal 36 in the I-V converter. The microprocessor 46 may determine a feedback signal that, when provided to the I-V converter 38, causes the I-V converter 38 to largely exclude the ambient light component of the photocurrent 36 in a variety of manners, as discussed below.

A digital-to-analog converter (DAC) 56 may convert the digital value of the feedback signal to an analog value, and may provide the analog value to ambient light cancellation circuitry 58. The ambient light cancellation circuitry 58 may generate a corresponding feedback signal 60 that may be provided to the I-V converter 38. As noted above, the feedback signal 60 may cause the output voltage 40 of the I-V converter 38 to include substantially only emitted light that has passed through the patient 30 and detected by the detector 22. Also, the microprocessor 46 may control the LED drive and/or switch 32 via the DAC 56, which may be a multi-channel DAC to accommodate the signals provided to the LED drive and/or switch 32 and the ambient light cancellation circuitry 58.

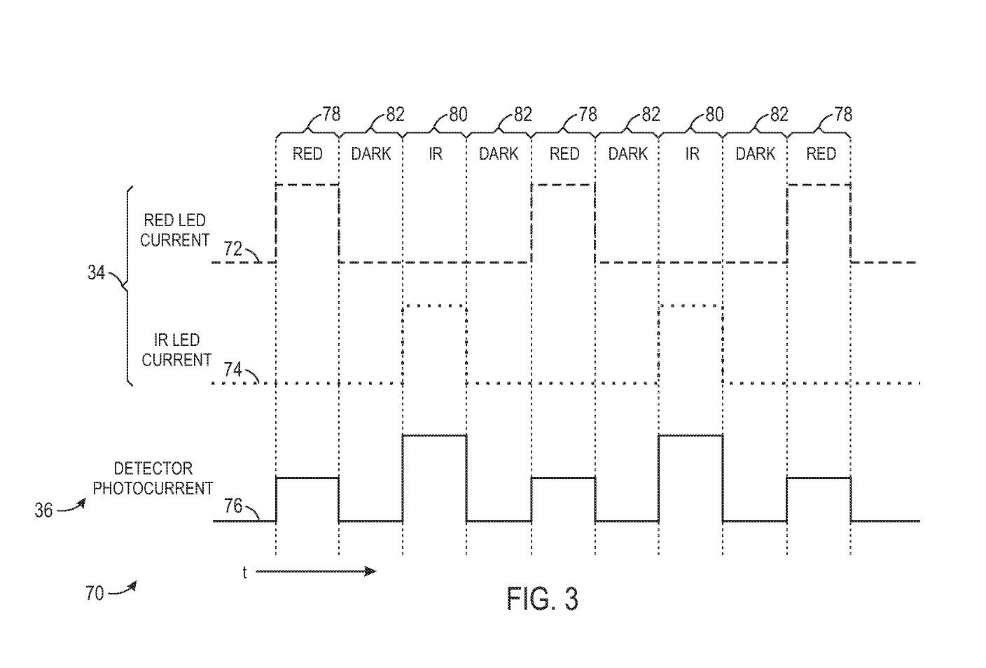

As noted above, to determine the feedback signal 60, the photocurrent signal 36 may be sampled when the emitter 20 is not emitting any light into the patient 30. Thus, the LED current signals 34 generally may include at least one dark period during which the ambient light component of the photocurrent 36 may be sampled. One embodiment of such LED current signals 34 and a resulting photocurrent signal 36 appear in a timing diagram 70 of FIG. 3. In the timing diagram 70, a curve 72 represents a first of the LED current signals 34 corresponding to a red wavelength LED, a curve 74 represents a second of the LED current signals 34 corresponding to an infrared (IR) wavelength, and a curve 76 represents the photocurrent signal 36.

As illustrated, the LED current signals 34 may cause the various LEDs of the emitter 20 to become operative at certain times. For example, during red periods 78, the curve 72 indicates that the first of the LED current signals 34 provides an operating current to red wavelength LEDs in the emitter 20, which subsequently emit red wavelength light into the patient 30. The detector 22 may accordingly detect an amount of red light passing through or reflected by the patient 30, which may relate to certain characteristics of the tissue of the patient 30 when compared to the IR light (e.g., the blood-oxygen saturation of hemoglobin in arterial blood, the volume of individual blood pulsations supplying the tissue, and/or the rate of blood pulsations). The red light detected by the detector 22 is represented by an increase in the photocurrent signal 36 during the red period 78, as illustrated by the curve 76.

Similarly, during IR periods 80, the curve 74 indicates that the second of the LED current signals 34 provides an operating current to IR wavelength LEDs in the emitter 20, which subsequently emit IR wavelength light into the patient 30. During the IR period 80, the detector 22 may detect an amount of IR light passing through or reflected by the patient 30, which may relate to certain characteristics of the tissue of the patient 30 as noted above. The IR light detected by the detector 22 is represented by an increase in the photocurrent signal 36 during the IR period 80, as illustrated by the curve 76.

During dark periods 82, none of the LEDs of the emitter 20 are operative. Thus, during such dark periods 82, the photocurrent 36 may only represent the amount of ambient light detected by the detector 22, as illustrated by the curve 76. Such a baseline ambient light component of the photocurrent 36 may be sampled and used to determine the feedback signal 60 applied to the I-V converter 38. Various methods for determining the feedback signal 60 are described in greater detail below.

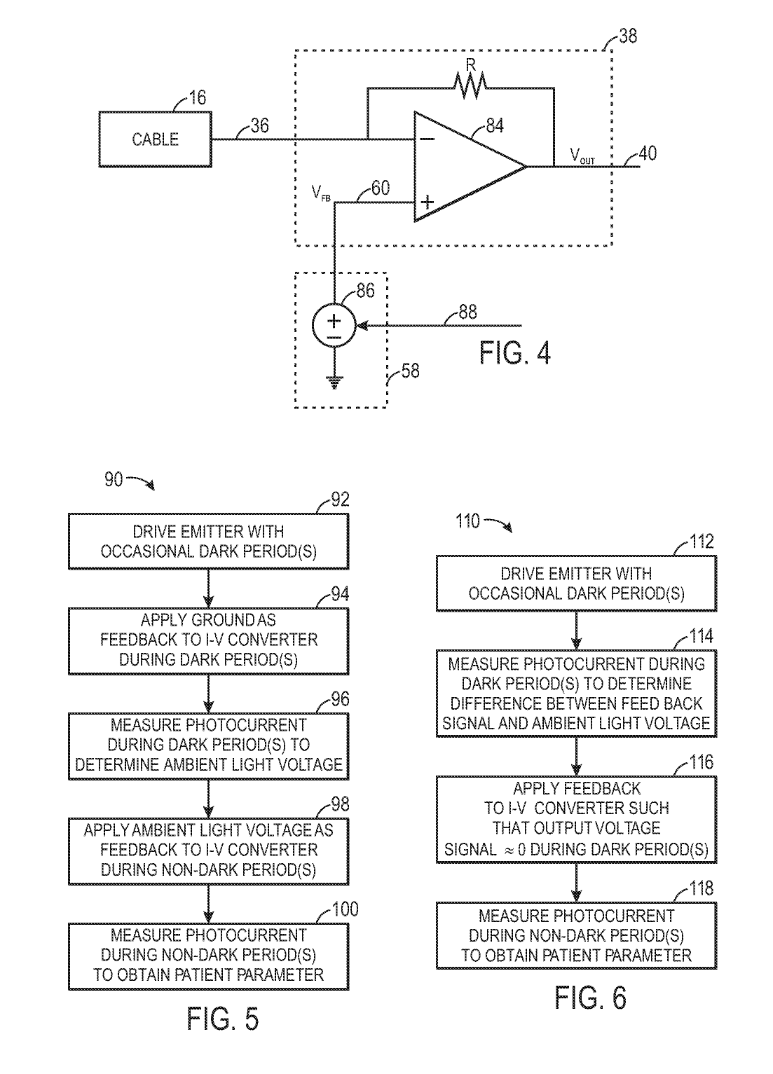

The application of the feedback signal 60 to the I-V converter 38 may prevent the ambient light component of the photocurrent 36 from being amplified by the I-V converter 38 during the red periods 78 and the IR periods 80. Thus, when the feedback signal 60 is applied to the I-V converter 38, substantially only the components of interest may be amplified and represented in the output voltage signal 40. Indeed, as illustrated in FIG. 4, the I-V converter 38 may include a transimpedance amplifier formed by an operational amplifier (op amp) 84 with negative feedback through a transimpedance R. From the cable 16, the photocurrent 36 may couple to the inverting (−) junction of the op amp 84 and output of the op amp 84, separated by the transimpedance R. Rather than coupling the summing non-inverting (+) junction of the op amp 84 to ground, the non-inverting junction (+) may be coupled to the feedback signal 60. As illustrated, the ambient light cancellation circuitry 58 may supply the feedback signal 60 and may include a voltage source 86 controlled by a control signal 88. The control signal 88, provided by the microprocessor 46 via the DAC 56, may cause the ambient light cancellation circuitry 58 to provide the feedback signal 60 as determined by the microprocessor 46.

The photocurrent 36 may pass across the transimpedance R and into the output node of the op amp 84. Since the op amp 84 will actively cause the inverting (−) node to virtually match the voltage on the non-inverting (+) node, the output voltage signal 40 may be represented by the relationship V_{OUT}=V_{FB}−IR, where V_{OUT} represents the output voltage signal 40, I represents the photocurrent signal 36, R represents the transimpedance of the I-V converter 38, and V_{FB} represents the feedback signal 60. Both the gain and the amount of thermal Johnson noise of the I-V converter 38 are affected by the transimpedance R. That is, the gain may be directly proportional to the transimpedance R, while the Johnson noise may be proportional to the square root of the transimpedance R. As such, when the transimpedance R is 1 MΩ rather than 100 kΩ (an increase of a factor of 10), the signal-to-noise ratio (SNR) of the I-V converter 38 may improve by a factor of \sqrt{10}, or approximately 3.

The higher SNR afforded by the higher transimpedance R could be negated by the higher gain provided by the higher transimpedance R unless the feedback signal 60 is applied. In particular, converting the photocurrent signal 36 into the output voltage signal 40 using the higher gain could cause the I-V converter 38 to operate outside of the signal saturation region of the I-V converter 38, which may distort the output voltage signal 40 and which becomes more likely at higher gains. To avoid such distortion, the feedback signal 60 may be determined such that the I-V converter 38 amplifies substantially only the components of the photocurrent 36 beyond the baseline ambient light component of the photocurrent 36 during the red period 78 and the IR period 80.

The feedback signal 60 applied during the red periods 78 and the IR periods 80 may be determined in a variety of ways. A flowchart 90 of FIG. 5 represents one embodiment of operating the medical sensor system 10 to obtain physiological parameters of the patient 30 involving one such manner of determining the feedback signal 60. The flowchart 90 may begin when the LED drive and/or switch 32 of patient monitor 12 drives the emitter 20 such that during the dark periods 82, the emitter 20 does not emit any light (block 92). As noted above, during these times substantially all of the light detected by the detector 22 may be ambient light. Thus, during one or more such dark periods 82, the microprocessor 46 may cause the ambient light cancellation circuitry 58 to tie the feedback signal 60 to ground (block 94), and the photocurrent 36 may be converted by the I-V converter 38 into the output voltage signal 40 before being analyzed by the microprocessor 46 (block 96). The output voltage signal 40 obtained during the one or more dark periods 82 while the feedback signal 60 is set to a ground voltage may represent a baseline ambient light voltage of the photocurrent 36. Thus, the microprocessor 46 may determine the feedback signal 60 to be applied during non-dark periods (e.g., the red period 78 and/or IR period 80) to equal such an output voltage signal 40. If multiple dark periods 82 are considered, the microprocessor 46 may average the output voltage signals 40 obtained during the multiple dark periods 82.

Thereafter, during the non-dark periods (e.g., the red periods 78 and IR periods 80), the microprocessor 46 may cause the ambient light cancellation circuitry 58 to provide the feedback signal 60 as equal to the determined baseline ambient light voltage (block 98). When the photocurrent 36 is measured using the I-V converter 38 during the non-dark periods to obtain the output voltage signal 40, which the microprocessor 46 and/or DSP 48 may use to determine the physiological parameter of the patient 30 (block 100), the output voltage signal 40 may substantially exclude ambient light noise. Moreover, since the output voltage signal 40 may include substantially only the components of interest of the photocurrent 36, the I-V converter 38 may be much more likely to operate within a saturation region, ensuring that the output voltage signal 40 is not distorted as a result. The feedback signal 60 may thus also enable the use of higher transimpedances R, and thus to obtain higher I-V converter 38 SNR. The method of the flowchart 90 of FIG. 5 may repeat indefinitely, refining the determination of the feedback signal 60 during subsequent dark periods 82.

Another method of operating the medical sensor system 10 may involve determining the feedback signal 60 and applying the same feedback signal 60 across all periods of operation (e.g., the red periods 78, the IR periods 80, and the dark periods 82), but varying the feedback signal 60 depending on the output voltage signal 40 obtained during the dark periods 82. A flowchart 110 of FIG. 6 may represent an embodiment of such a method. The flowchart 110 may begin when the LED drive and/or switch 32 of patient monitor 12 drives the emitter 20 such that during the dark periods 82, the emitter 20 does not emit any light (block 112). As noted above, during these times substantially all of the light detected by the detector 22 may be ambient light. The microprocessor 46 may cause the ambient light cancellation circuitry 58 to apply the feedback signal 60 at a starting voltage and, during the dark periods 82, the photocurrent 36 may be converted by the I-V converter 38 into the output voltage signal 40 and analyzed by the microprocessor 46 (block 114).

The output voltage signal 40 that is obtained during the one or more dark periods 82 while the feedback signal 60 is applied may represent an error signal. That is, the closer the feedback signal 60 is to the baseline ambient light voltage, the closer the output voltage signal 40 will be to 0V during the dark periods 82. Thus, the microprocessor 46 may apply any suitable signal control technique to adjust the feedback signal 60 higher or lower depending on the output voltage signal 40 obtained during the dark periods 82, such that during the dark periods 82, the output voltage signal 40 is approximately 0V (block 116). When the output voltage signal 40 is approximately 0V during the dark periods 82, the feedback signal 60 may be understood to equal approximately the ambient light voltage, and thus the I-V converter 38 may be understood effectively to cancel the ambient light component of the photocurrent 36. In some embodiments, the microprocessor 46 may consider multiple dark periods 82 and averaging the obtained output voltage signals 40 before adjusting the feedback signal 60.

The I-V converter 38 may measure the photocurrent 36 with the feedback signal 60 also applied to the I-V converter 38 during the non-dark periods. Based on the resulting output voltage signal 40, the microprocessor 46 and/or the DSP 48 may determine the physiological parameter of the patient 30 (block 118). Since the feedback signal 60 may cause the I-V converter 38 to substantially exclude ambient light noise, the output voltage signal 40 may include substantially only the components of interest of the photocurrent 36. Moreover, as noted above, the I-V converter 38 may be much more likely to operate within a saturation region, ensuring that the output voltage signal 40 is not distorted due to operation outside the saturation region. The application of the feedback signal 60 may also enable the use of higher transimpedances R, and thus to obtain higher I-V converter 38 SNR. The method of the flowchart 110 of FIG. 6 may repeat indefinitely, refining the determination of the feedback signal 60 during subsequent dark periods 82.

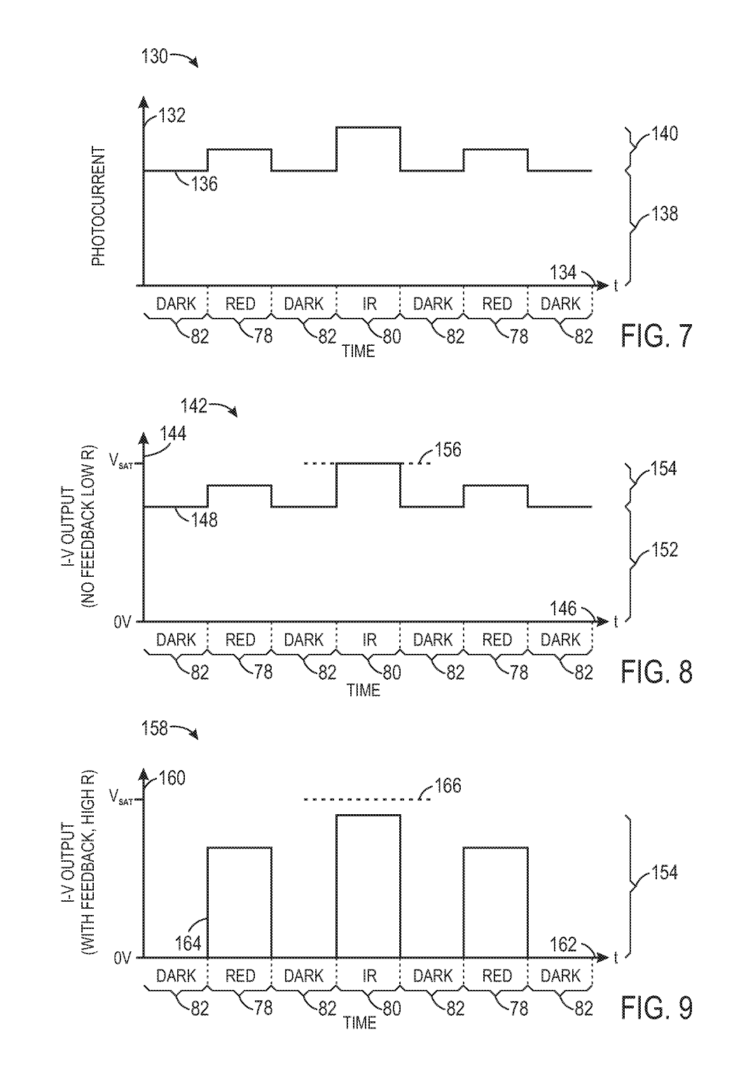

Sample plots representing certain benefits of the present disclosure appear in FIGS. 7-9. In particular, FIG. 7 represents an exemplary photocurrent 36 over time, FIG. 8 represents a corresponding output voltage signal 40 obtained when the disclosed techniques are not employed, and FIG. 9 represents a corresponding output voltage signal 40 obtained when the disclosed techniques are employed. Turning to FIG. 7, a plot 130 models a photocurrent 36 (ordinate 132) over time (abscissa 134). A curve 136 illustrates the manner in which the photocurrent 36 may change over various periods (e.g., the red periods 78, the IR periods 80, and the dark periods 82). All periods include an ambient light component 138, which represents the baseline amount of ambient light detected by the detector 22. However, only during the periods in which the emitter 20 is emitting light (e.g., the red period 78 and the IR period 80) does a component of interest 140 of the photocurrent 36 vary.

When the photocurrent 36 illustrated by the plot 130 is amplified by an I-V converter 38 that does not employ ambient light cancellation feedback in the manners described above (and/or includes a relatively low transimpedance R), the resulting output voltage signal 40 may suffer from certain problems. For example, a plot 142 of FIG. 8 models the conversion of the photocurrent 36 of FIG. 7 into an output voltage signal 40 in volts (ordinate 144) over time (abscissa 146) when the feedback signal 60 is not applied to the I-V converter 38. The I-V converter 38 modeled in the plot 142 may also include a relatively low transimpedance R, and thus a relatively low SNR with respect to Johnson noise. As apparent from a curve 148, which represents the changing output voltage signal 40 over time, a significant portion of the output voltage signal 40 could be representative of the ambient light component 138 of the photocurrent 36. Indeed, such an ambient light voltage component 152 of the output voltage signal 40, which may be present through all periods, could greatly exceed a component of interest 154 of the output voltage signal 40, which may be present only when the emitter 20 is emitting light (e.g., the red period 78 and the IR period 80).

Although the LP filter 42 generally may remove the ambient light voltage component 152, leaving substantially only the component of interest 154, the gain of the I-V converter 38 may remain relatively low to prevent the I-V converter 38 from operating beyond the region of saturation. As such, the SNR of the I-V converter 38 also may remain relatively lower. Indeed, as indicated by a numeral 156, if the transimpedance R is too high, the output voltage signal 40 may exceed a saturation voltage V_{SAT} of the I-V converter 38, resulting in signal distortion.

On the other hand, when the feedback signal 60 is determined and applied as disclosed herein, the I-V converter 38 may employ a higher transimpedance R without such problems. Accordingly, the SNR of the I-V converter 38 may be correspondingly improved. FIG. 9 illustrates a plot 158 modeling the conversion of the photocurrent 36 of FIG. 7 into an output voltage signal 40 in volts (ordinate 160) over time (abscissa 162) when the techniques described herein are employed. When the feedback signal 60 is applied as discussed above, the ambient light component 138 of the photocurrent 36 may be largely cancelled. Thus, as illustrated by a curve 164 representing the output voltage signal 40, the output voltage signal 40 may substantially only include the component of interest 154. Moreover, since the I-V converter 38 may employ a higher transimpedance R without operating beyond a signal saturation region (numeral 166), the I-V converter 38 may also operate with a higher SNR with respect to Johnson noise.

As apparent from the curve 164, the component of interest 154 may be substantially more amplified when the I-V converter 38 employs a higher transimpedance R and thus achieves a higher gain and a higher SNR. With such improvements in signal processing, the photocurrent 36 could be significantly reduced while still achieving results comparable to those achieved with conventional designs and higher photocurrents 36. Indeed, with the disclosed techniques, the emitters 20 may be driven with less current and less light may be emitted into the patient 30 and/or the emitters 20 may be smaller and/or weaker while still providing useful physiological data about the patient 30.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

What is claimed is:

1. A patient monitor comprising:
drive circuitry configured to cause an emitter of a medical sensor to emit light into a patient tissue, wherein the emitter does not emit the light during a dark period;
a current-to-voltage converter configured to convert a photocurrent signal generated by a detector of the medical sensor in response to light received by the detector to obtain an output voltage signal, wherein the received light includes emitted light that has interacted with the patient tissue and ambient light when the emitter is emitting the light and wherein the received light includes the ambient light during the dark period; and
feedback circuitry configured to provide a feedback signal to the current-to-voltage converter, wherein the feedback signal causes the current-to-voltage converter to substantially remove a component of the output voltage signal that corresponds to the ambient light when the emitter is emitting the light.

2. The patient monitor of claim 1, wherein the current-to-voltage converter comprises an operational amplifier and wherein the feedback circuitry is configured to provide the feedback signal to a non-inverting junction of the operational amplifier.

3. The patient monitor of claim 1, wherein the feedback circuitry is configured to provide the feedback signal both during the dark period and when the emitter is emitting the light.

4. The patient monitor of claim 3, wherein the feedback signal is configured to cause the current-to-voltage converter to output approximately 0V during the dark period.

5. The patient monitor of claim 1, wherein the feedback circuitry is configured to provide a ground voltage to the current-to-voltage converter during the dark period rather than the feedback signal.

6. The patient monitor of claim 5, wherein the feedback signal approximately equals the output voltage signal obtained during the dark period.

7. The patient monitor of claim 1, comprising processing circuitry configured to determine the feedback signal based at least in part on the output voltage signal obtained during the dark period.

8. The patient monitor of claim 7, wherein the processing circuitry is configured to determine the feedback signal based at least in part on a plurality of values of the output voltage signal obtained during a respective plurality of dark periods.

9. The patient monitor of claim 1, comprising patient parameter determination circuitry configured to determine a patient parameter based at least in part on the output voltage signal obtained when the emitter is emitting light.

10. A method comprising:
measuring, using a current-to-voltage converter, a photocurrent signal from a detector of a medical sensor while an emitter of the medical sensor is not emitting light to obtain a first output voltage signal, wherein the first output voltage signal corresponds primarily to a noise component of the photocurrent signal;
applying a feedback signal to the current-to-voltage converter, wherein the feedback signal is based at least in part on the first output voltage signal; and
measuring, using the current-to-voltage converter while the feedback signal is applied, the photocurrent signal while the emitter of the medical sensor is emitting light to obtain a second output voltage signal, wherein the feedback signal causes the current-to-voltage converter to output the second output voltage signal such that the second output voltage signal corresponds substantially only to a component of the photocurrent signal other than the noise component.

11. The method of claim 10, wherein the first output voltage signal corresponds primarily to a component of the photocurrent signal representing ambient light detected by the detector of the medical sensor.

12. The method of claim 10, wherein the second output voltage signal corresponds primarily to a component of the photocurrent signal representing light emitted by the emitter and detected by the detector of the medical sensor.

13. The method of claim 10, wherein the feedback signal is applied to a non-inverting junction of an operational amplifier of the current-to-voltage converter.

14. The method of claim 10, wherein the feedback signal causes the first output voltage signal to equal approximately 0V when the photocurrent signal is measured while the emitter of the medical sensor is not emitting light.

15. The method of claim 10, wherein the feedback signal is not applied when the photocurrent signal is measured while the emitter of the medical sensor is not emitting light and wherein the feedback signal applied when the emitter of the medical sensor is emitting light is approximately equal to the first output voltage signal.

16. A system comprising:
a medical sensor comprising:
an emitter configured to emit light into a patient based on emitter driving signals; and
a detector configured to detect light and to generate a detector signal based on the detected light, wherein the detector signal includes a first component based on emitted light that passes through the patient and a second component based on ambient light; and
a patient monitor comprising:
emitter driving circuitry configured to generate the emitter driving signals, wherein the emitter driving signals are configured to cause the emitter not to emit light during at least one dark period;
signal amplifier circuitry configured to amplify substantially only the first component of the detector signal based on a feedback signal when the emitter is emitting light into the patient; and
feedback signal determination circuitry configured to determine the feedback signal based at least in part on the output signal obtained during the at least one dark period.

17. The system of claim 16, wherein the signal amplifier circuitry comprises a transimpedance amplifier configured to amplify the first component of the detector signal within a signal saturation region of the transimpedance amplifier.

18. The system of claim 17, wherein the transimpedance amplifier has a transimpedance greater than 100 kΩ.

19. The system of claim 17, wherein the transimpedance amplifier has a transimpedance greater than 1 MΩ.

20. The system of claim 16, wherein the feedback signal determination circuitry comprises a processor configured to determine a digital value of the feedback signal, wherein the patient monitor comprises a digital to analog converter configured to transform the digital value of the feedback signal to the feedback signal and wherein the patient monitor comprises feedback circuitry configured to apply the feedback signal to the signal amplifier circuitry.
